(19) United States
(12) Patent Application Publication
(10) Pub. No.: US 2023/0320690 A1
(43) Pub. Date: Oct. 12, 2023

(54) ANALYSIS DEVICE

(71) Applicant: NEC Corporation, Minato-ku, Tokyo (JP)

(72) Inventors: Masao Higuchi, Tokyo (JP); Mitsuru Noma, Tokyo (JP); Reishi Kondo, Tokyo (JP); Yumi Arai, Tokyo (JP)

(73) Assignee: NEC Corporation, Minato-ku, Tokyo (JP)

(21) Appl. No.: 18/021,430

(22) PCT Filed: Aug. 25, 2020
(86) PCT No.: PCT/JP2020/032060

§ 371 Date: Feb. 15, 2023

(51) Int. Cl. A61B 7/00; G16H 50/20; G16H 50/30; A61B 7/04

(52) U.S. Cl. CPC A61B 7/003; G16H 50/20; G16H 50/30; A61B 7/04

(57) ABSTRACT

An analysis device includes a detection unit that detects abnormality in lung sounds for each of auscultation positions on the basis of time-series acoustic signals including lung sounds of each of the auscultation positions; a determination unit that determines severity of heart failure of a patient on the basis of a detection result of abnormality in the lung sounds of each of the auscultation positions detected by the detection unit and condition information representing a condition of the patient; and an output unit that decides whether or not to output predetermined information to an external device on the basis of a result of determination by the determination unit, and performs output corresponding to a result of decision.

### TECHNICAL FIELD

The present invention relates to an analysis device, an analysis method, and a storage medium.

### BACKGROUND ART

Heart failure is a clinical syndrome in which as a result that cardiac dysfunction, that is, an organic and/or functional dysfunction, occurred in the heart and compensation mechanism of a heart pump function failed, dyspnea, malaise, or an edema appears, which is accompanied by a drop of exercise tolerability. A patient who suffered from heart failure always has a risk of exacerbation even though the patient has been treated and reached remission. When acute exacerbation occurs in the patient due to excessive water or salt intake, forgetting to take medicines, too much exercise, and the like, the patient must be hospitalized again. Therefore, it is important to prevent acute exacerbation by finding heart failure exacerbation of a patient discharged from hospital in an early stage and giving treatment intervention.

One method of diagnosing heart failure is a lung sound examination by auscultation. Such an examination is a method usable for diagnosing health condition of lungs and also heart failure, in a safe and easy manner. However, it is difficult for those other than skilled medical specialists to obtain a detailed and accurate diagnosis result. Therefore, in the rounds by general nurses or caring staff and in the visiting care sites, it is impossible to obtain a detailed diagnosis.

In order to cope with such a problem, a system has been proposed. The system automatically determines presence or absence of abnormal sounds called adventitious sounds in the lung sounds collected by an electronic stethoscope (for example, see Patent Literatures 1 to 4 and 6). Another system has also been proposed. This system is for detecting abnormality by comparing biological sound data of a patient collected by an electronic stethoscope with normal time data and abnormal time data of the patient acquired in advance (for example, Patent Literature 5).

Further, as related art, Patent Literature 7 has been known. Patent Literature 7 discloses a system for determining a doctor, who gives medical care, to be recommended to a patient based on the information of the disease condition input by the patient, and transmitting the information of the doctor, who gives medical care, to a patient's terminal.

Patent Literature 1: JP 2014-4018 A

Patent Literature 2: JP 2002-538921 A

Patent Literature 3: JP 2017-536905 A

Patent Literature 4: WO 2010/044452 A

Patent Literature 5: JP 2008-113936 A

Patent Literature 6: JP 4849424 B

Patent Literature 7: JP 2019-79501 A

Patent Literature 8: JP 2007-190081 A

### SUMMARY

When a person not having professional knowledge such as a patient takes an examination by using the art as described in Patent Literatures 1 to 6, there is a problem that it is difficult to efficiently share the examination result with a medical professional. Further, as described in Patent Literature 7, in the case of a configuration of transmitting disease condition information input by the patient, the examination result is transmitted to a medical professional at random, which makes it difficult to efficiently share the examination result. This causes a problem that it is difficult to efficiently share the examination result with a medical professional as described above, for example.

In view of the above, an object of the present invention is to provide an analysis device, an analysis method, and a storage medium capable of solving a problem that it is difficult to efficiently share an examination result with medical professionals.

An analysis device, according to one aspect of the present invention, is configured to include
a detection unit that detects abnormality in lung sounds for each of auscultation positions on the basis of time-series acoustic signals including lung sounds of each of the auscultation positions;
a determination unit that determines severity of heart failure of a patient on the basis of a detection result of abnormality in the lung sounds of each of the auscultation positions detected by the detection unit and condition information representing a condition of the patient; and
an output unit that decides whether or not to output predetermined information to an external device on the basis of a result of the determination by the determination unit, and performs output corresponding to a result of the decision.

Further, an analysis method, according to another aspect of the present invention, is configured to include, by an information processing device:
detecting abnormality in lung sounds for each of auscultation positions on the basis of time-series acoustic signals including lung sounds of each of the auscultation positions;
determining severity of heart failure of a patient on the basis of a detection result of abnormality in the lung sounds of each of the auscultation positions detected and condition information representing a condition of the patient; and
deciding whether or not to output predetermined information to an external device on the basis of a result of the determination, and performing output corresponding to a result of the decision.

Further, a storage medium, according to another aspect of the present invention, is a computer-readable medium storing thereon a program for implementing:
a detection unit that detects abnormality in lung sounds for each of auscultation positions on the basis of time-series acoustic signals including lung sounds of each of the auscultation positions;
a determination unit that determines severity of heart failure of a patient on the basis of a detection result of abnormality in the lung sounds of each of the auscultation positions detected by the detection unit and condition information representing a condition of the patient; and
an output unit that decides whether or not to output predetermined information to an external device on the basis of a result of the determination by the determination unit, and performs output corresponding to a result of the decision.

With the configurations described above, the present invention enables an examination result to be shared with medical professionals efficiently.

### BRIEF DESCRIPTION OF DRAWINGS

FIG. 1 is a diagram illustrating an exemplary configuration of the entire analysis system according to a first exemplary embodiment of the present invention.

FIG. 2 is a block diagram illustrating an exemplary configuration of the analysis device illustrated in FIG. 1.

FIG. 3 illustrates an example of lung sound data.

FIG. 4 is a diagram for explaining exemplary auscultation positions.

FIG. 5 is a diagram for explaining exemplary auscultation positions.

FIG. 6 illustrates an example of analysis result information.

FIG. 7 illustrates an example of severity determination information.

FIG. 8 illustrates an example of severity information.

FIG. 9 illustrates exemplary information included in personal condition information.

FIG. 10 illustrates an example of output information.

FIG. 11 is a block diagram illustrating an exemplary configuration of the processing device illustrated in FIG. 1.

FIG. 12 is a flowchart illustrating an exemplary operation of the analysis device.

FIG. 13 illustrates an exemplary configuration of an analysis system according to a second exemplary embodiment of the present invention.

FIG. 14 illustrates an exemplary hardware configuration of an analysis device according to a third exemplary embodiment of the present invention.

FIG. 15 is a block diagram illustrating an exemplary configuration of an analysis device.

### EXEMPLARY EMBODIMENTS

### First Exemplary Embodiment

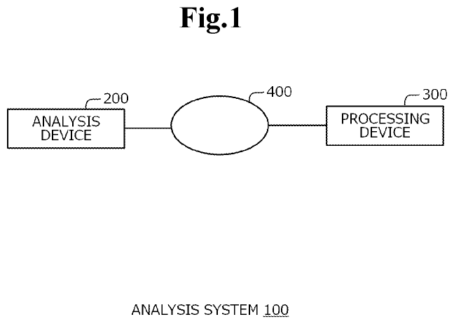

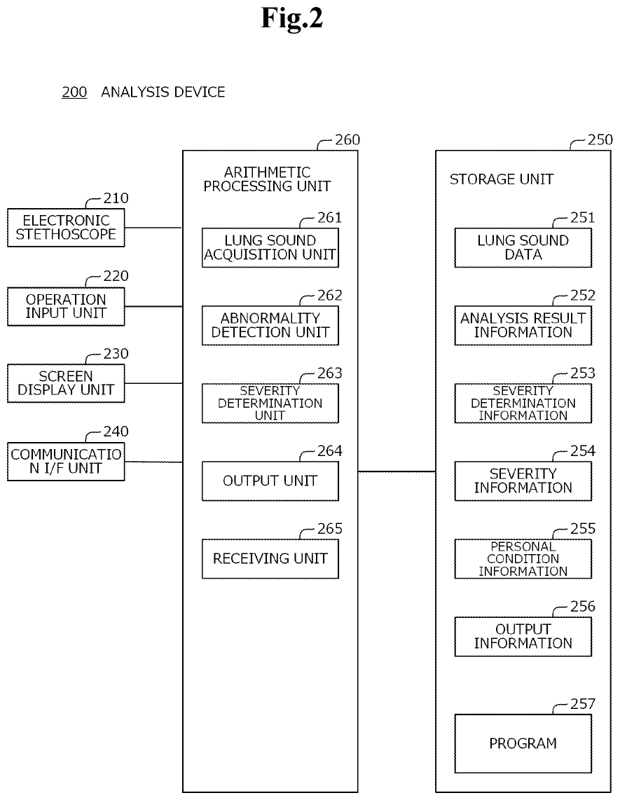

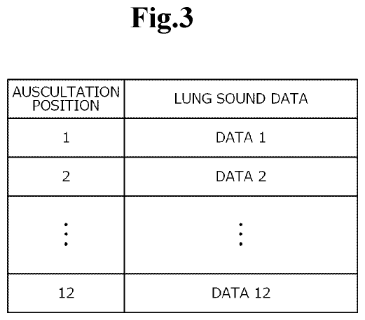

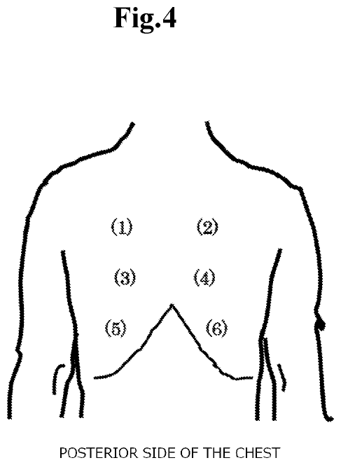

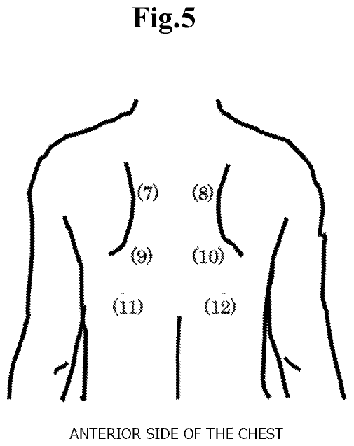

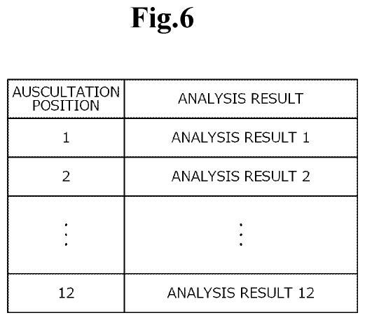

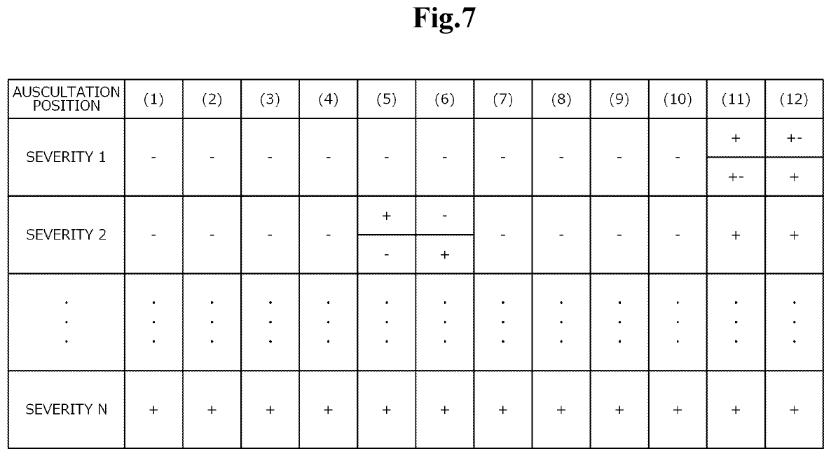

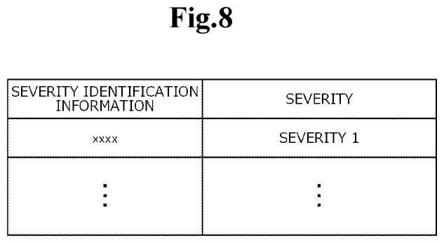

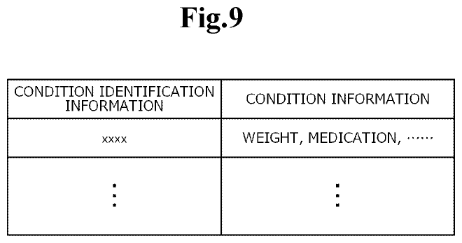

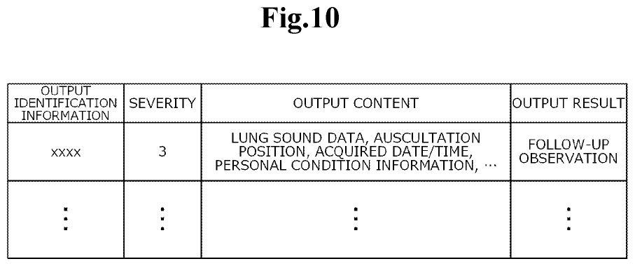

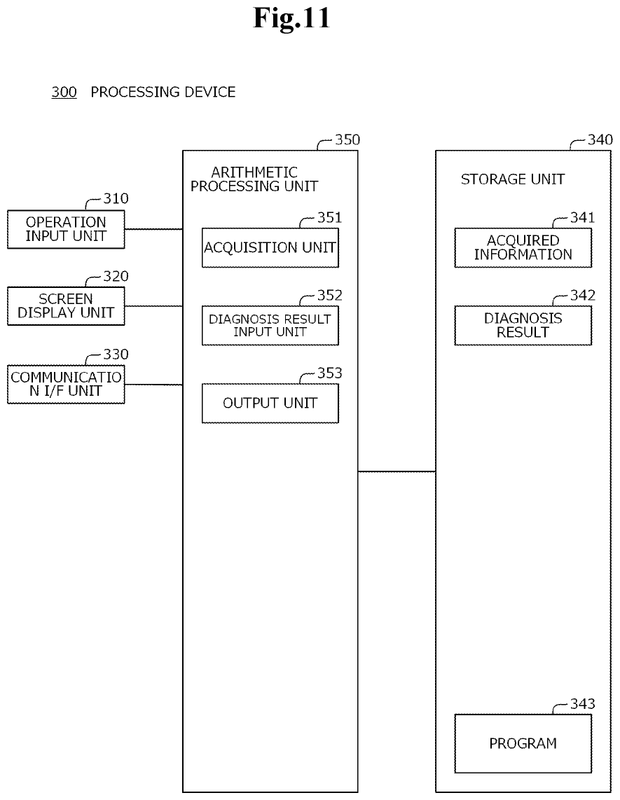

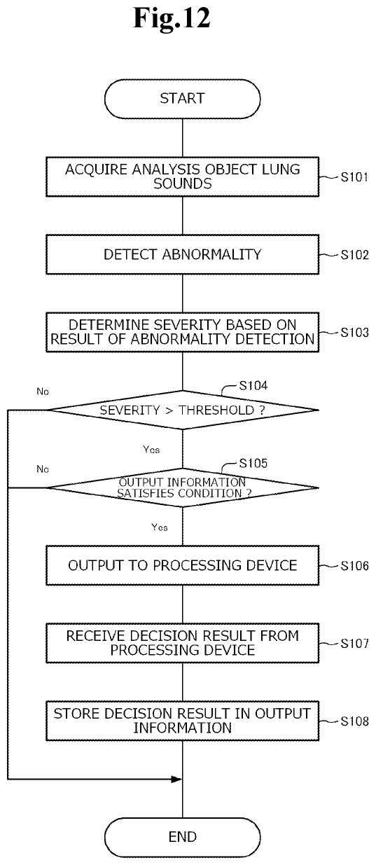

A first exemplary embodiment of the present invention will be described with reference to FIGS. 1 to 12. FIG. 1 is a diagram illustrating an exemplary configuration of the entire analysis system 100. FIG. 2 is a block diagram illustrating an exemplary configuration of an analysis device 200. FIG. 3 illustrates an example of lung sound data 251. FIGS. 4 and 5 are diagrams for explaining exemplary auscultation positions. FIG. 6 illustrates an example of analysis result information 252. FIG. 7 illustrates an example of severity determination information 253. FIG. 8 illustrates an example of severity information 254. FIG. 9 illustrates exemplary information included in personal condition information 255. FIG. 10 illustrates an example of output information 256. FIG. 11 is a block diagram illustrating an exemplary configuration of a processing device 300. FIG. 12 is a flowchart illustrating an exemplary operation of the analysis device 200.

In the first exemplary embodiment of the present invention, the analysis system 100 having the analysis device 200 that transmits predetermined information to a processing device 300 according to a result of analyzing lung sounds will be described. As described below, the analysis device 200 acquires lung sounds from a plurality of positions on the posterior side and the anterior side of the chest, and detects abnormality in each position based on the acquired lung sounds. The analysis device 200 also determines the severity of heart failure based on the detected result. Then, the analysis device 200 determines whether or not to output predetermined information to the processing device 300, based on the determined severity.

The analysis device 200 also includes output information 156 including results output to the processing device 300 in the past. When deciding whether or not to output information to the processing device 300, the analysis device 200 can refer to the information included in the output information 256.

FIG. 1 illustrates an exemplary configuration of the entire analysis system 100. Referring to FIG. 1, the analysis system 100 includes the analysis device 200 and the processing device 300. As illustrated in FIG. 1, the analysis device 200 and the processing device 300 are communicably connected with each other over a network 400. The analysis system 100 can include any number of analysis devices 200 and processing devices 300.

The analysis device 200 is an information processing device that decides whether or not to transmit predetermined information to the processing device 300, according to an analysis result of lung sounds acquired from a patient. The analysis device 200 may be a smartphone, a tablet terminal, a personal digital assistant (PDA), a laptop personal computer, or the like, for example. The analysis device 200 may be one other than that mentioned above as an example.

FIG. 2 illustrates an exemplary configuration of the analysis device 200. Referring to FIG. 2, the analysis device 200 includes, for example, an electronic stethoscope 210, an operation input unit 220, a screen display unit 230, a communication I/F unit 240, a storage unit 250, and an arithmetic processing unit 260, as main constituent elements.

The electronic stethoscope 210 acquires lung sounds of a patient. For example, when the chest piece of the stethoscope is attached to the posterior side or the anterior side of the chest of a patient, the electronic stethoscope 210 converts the lung sounds of the patient into digital signals, and transfers them to the arithmetic processing unit 260 in a wireless or wired manner.

The operation input unit 220 is configured of operation input devices such as a keyboard and a mouse. The operation input unit 220 detects operation by a user who uses the analysis device 200, and outputs it to the arithmetic processing unit 260. The users may include a medical professional such as a doctor or a nurse, caring staff such as a care worker, or family of the patient, besides the patient.

The screen display unit 230 is configured of a screen display device such as a liquid crystal display (LCD). The screen display unit 230 can display, on the screen, various types of information such as an analysis result, in response to an instruction from the arithmetic processing unit 260.

The communication I/F unit 240 is configured of a data communication circuit. The communication I/F unit 240 performs data communication with various external devices such as the processing device 300 connected via a wired or wireless manner.

The storage unit 250 is a storage device such as a hard disk or a memory. The storage unit 250 stores therein processing information and a program 257 required for various types of processing performed in the arithmetic processing unit 260. The program 257 is read and executed by the arithmetic processing unit 260 to thereby implement various processing units. The program 257 is read in advance from an external device or a storage medium via the data input/output function of the communication I/F unit 240 or the like, and is stored in the storage unit 250. Main information stored in the storage unit 250 includes, for example, lung sound data 251, analysis result information 252, severity determination information 253, severity information 254, personal condition information 255, and output information 256.

The lung sound data 251 represents lung sound data of each auscultation position. FIG. 3 illustrates an example of information included in the lung sound data 251. Referring to FIG. 3, the lung sound data 251 includes lung sound data of each auscultation position, for example.

The information as illustrated in FIG. 3 included in the lung sound data 251 is created each time analysis is performed using the analysis device 200 or each time auscultation is performed using the electronic stethoscope 210, for example. The lung sound data 251 may be a combination of data identification information corresponding to the auscultation date/time or the like and the information as illustrated in FIG. 3.

Here, the field of auscultation position in the information included in the lung sound data 251 indicates an approximate position of the body of a patient to which the chest piece of the electronic stethoscope 210 is applied for auscultation of the lung sounds. That is, the auscultation position is a position for acquiring the lung sounds. For example, in the example of FIG. 3, twelve positions in total from an auscultation position (1) to an auscultation position (12) are set (in FIG. 3, auscultation positions (3) to (11) are omitted).

FIG. 4 is a schematic diagram for explaining examples of the auscultation positions (1) to (6), and FIG. 5 is a schematic diagram for explaining examples of the auscultation positions (7) to (12). Referring to FIG. 4, the auscultation positions (1) and (2) are set at left and right of the upper lung field on the posterior side of the chest, for example. The auscultation positions (3) and (4) are set at left and right of the middle lung field on the posterior side of the chest, for example. The auscultation positions (5) and (6) are set at left and right of the lower lung field on the posterior side of the chest, for example. Referring to FIG. 5, the auscultation positions (7) and (8) are set at left and right of the upper lung field on the anterior side of the chest. The auscultation positions (9) and (10) are set at left and right of the middle lung field on the anterior side of the chest. The auscultation positions (11) and (12) are set at left and right of the lower lung field on the anterior side of the chest. As described above, the auscultation positions are set in advance, for example.

Note that the auscultation positions are not limited to the number and the positions described above. For example, not only the posterior side and the anterior side of the chest, it is also possible to set auscultation positions in the upper lung field, the middle lung field, and the lower lung field of the left and right side chest parts to have eighteen positions in total. Alternatively, some of the above-described auscultation positions may be excluded. For example, it is possible to exclude the auscultation positions (3) to (6), (9), and (10) to thereby limit the positions to six positions in total, that is, the auscultation positions (1), (2), (7), (8), (11), and (12).

Further, in the field of lung sound data of the lung sound data 251, digital time-series acoustic signals including lung sounds obtained by the electronic stethoscope 210 at an auscultation position are recorded. The posture of the patient at the time of auscultation is roughly classified into a lying position and a sitting position. The auscultation of the posterior side of the chest and the anterior side of the chest is generally performed in a sitting position. The signal length of one piece of lung sound data (for example, data 1) may have any length. One piece of lung sound data may include signals of continuous N times of respiration of a patient. Here, N represents a positive integer of 1 or larger. The lung sound data may be signals in which processing such as removal of time-series acoustic signals in a period of a pause phase, noise removal, and application of respiration timing is performed on the time-series acoustic signals obtained from the electronic stethoscope 210.

The analysis result information 252 indicates a result of detecting abnormality based on the lung sound data 251 by an abnormality detection unit 262 to be described below. FIG. 6 illustrates an example of information included in the analysis result information 252. Referring to FIG. 6, the analysis result information 252 includes an analysis result of each auscultation position, for example.

The information as illustrated in FIG. 6 included in the analysis result information 252 is created each time analysis using the analysis device 200 is performed or each time analysis is performed on the lung sound data 251 by the abnormality detection unit 262, for example. The analysis result information 252 may be a combination of analysis result identification information corresponding to the date/time on which the abnormality detection unit 262 performed analysis or the like and the information as illustrated in FIG. 6.

In the analysis result field of the information included in the analysis result information 252, a result of mechanically analyzing the lung sound data by the abnormality detection unit 262, described below, is recorded. For example, in the analysis result field, a numerical value indicating whether or not the lung sound data is abnormal lung sound data is recorded. For example, the analysis result field may contain a binary value, that is, a value 0 indicating normal lung sounds or a value 1 indicating abnormal lung sounds. Alternatively, the analysis result field may contain a numerical value representing the abnormal degree of the lung sound data. Regarding the abnormal degree, an abnormal degree that is equal to or less than a preset threshold represents that the lung sound data is normal lung sounds, and an abnormal degree exceeding the threshold represents that the lung sound data is abnormal lung sounds.

The severity determination information 253 is information used to determine the severity by the severity determination unit 263 to be described below. The severity determination information 253 is, for example, acquired in advance by a method such as being read from an external device or a storage medium via the data input/output function of the communication I/F unit 240 or the like, being input through operation of the operation input unit 220 by the user, or the like, and is stored in the storage unit 250. The severity determination information 253 may be created at the timing when the patient is discharged from hospital. In other words, the severity determination information 253 may be created according to the condition of the patient at the time of discharge from hospital. In general, most of hospitalized heart failure patients leave hospital in a remission state after receiving a heart failure treatment. Therefore, the lung sounds at the time of discharge from hospital are normal in most patients. However, there is a case where a patient is discharged from hospital in a mild case due to the circumstances of the patient. By creating the severity determination information 253 according to the condition at the time of discharge from hospital, it is possible to perform determination according to the condition of the patient at the time of discharge from the hospital such as a patient who left the hospital in a remission state or a patient who left the hospital in a mild case. The severity determination information 253 may be created or updated at the timing when a patient attends hospital.

FIG. 7 illustrates an example of the severity determination information 253. Referring to FIG. 7, the severity determination information 253 is a table that includes, for example, a column corresponding to each of the auscultation positions (1) to (12) one to one, and a row corresponding to a degree of severity one to one. At an intersection between a column and a row, a + sign indicating that there is abnormality in the lung sounds or a − sign indicating that there is no abnormality in the lung sounds are set. For, example, in the example illustrated in FIG. 7, the table of the severity determination information 253 indicates that the severity is determined to be severity 0 when there is no abnormality in lung sounds at any auscultation positions. When there is abnormality in the lung sounds at at least one of the auscultation positions (11) and (12) set in the lower lung field in the anterior side of the chest and there is no abnormality in the lung sounds at the other auscultation positions (1) to (10), it is determined that the severity is 1. Further, when there is abnormality in the lung sounds at both the auscultation positions (11) and (12), there is abnormality in the lung sounds at either one of the auscultation positions (5) and (6) set in the lower lung field of the posterior side of the chest, and there is no abnormality in the lung sounds in the other auscultation positions (1) to (4) and (7) to (10), it is determined that the severity is 2. The severity N set in the last row means that there is abnormality in the lung sounds at all auscultation positions (1) to (12). In FIG. 6, although description of one or more degrees of severity is omitted between the severity 2 and the severity N, auscultation positions having abnormality in the lung sounds and auscultation positions not having abnormality in the lung sounds are also set for them. In the one or more degrees of severity between the severity 2 and the severity N, the number of auscultation positions at which there is abnormality in the lung sounds is four or larger and less than twelve, and the number increases as closer to the severity N.

As described above, in the severity determination information 253, the severity of heart failure is classified into N+1 classes from the severity 0 to the severity N, depending on the combination of presence or absence of abnormality in the lung sounds at the auscultation positions (1) to (12), for example. Here, the severity 0 is a state where no abnormal lung sound is heard. Therefore, it can be said that heart failure is remitted. The severity 1 is a condition in which abnormal lung sounds are heard only in the lower lung field of the anterior side of the chest. Therefore, heart failure is mild although not remitted, and is a condition in which some patients are discharged from hospital in such a condition. The severity 2 is a condition in which abnormal lung sounds are heard in one of the lower lung field of the posterior side of the chest in addition to the lower lung field of the anterior side of the chest. Therefore, it can be said that it is more severe than the severity 1. However, it still belongs to the mild case, so there is a high possibility of preventing re-hospitalization if it is treated appropriately at this point.

Note that the information shown in the severity determination information 253 is not limited to that illustrated in FIG. 7. The severity determination information 253 may indicate information other than that illustrated in FIG. 6. For example, the case where there is abnormality in lung sounds at at least one of the auscultation positions (11) and (12) set in the lower lung field of the anterior side of the chest and there is no abnormality in lung sounds at the other auscultation positions (1) to (10), and the case where there is abnormality in lung sounds at both of the auscultation positions (11) and (12), there is abnormality in lung sounds at at least one of the auscultation positions (5) and (6), and there is no abnormality in lung sounds at the other auscultation positions (1) to (4) and (7) to (10), may be set to be severity 1. The number of columns of the severity determination information 253 may correspond to the number of the auscultation positions previously set in the lung sound data 251.

Further, it is known that when rales are heard at the end of inspiration, it is mild, and when the rales are heard immediately after the start of inspiration, it is severe. Therefore, in addition to presence or absence of abnormal lung sounds at each auscultation position, the timing that the abnormal lung sounds are heard may be added to the determination table, and the severity of the heart failure may be determined according to the combination of an auscultation position, presence or absence of abnormal lung sounds, and the timing that the abnormal lung sounds are heard. In the severity determination information 253, the degrees of severity may be set corresponding to the type and the number of abnormal sounds such as rales having different characteristics (rough discontinuous rales, fine continuous rales). Further, in the severity determination information 253, the degrees of severity may be set corresponding to information that may be included in the personal condition information 255 such as the amount of weight increase of the patient. Further, the severity determination information 253 may be information in which the number of auscultation positions at which abnormal lung sounds are heard and the severity of heart failure of the patient are associated with each other, for example. For example, in the severity determination information 253, the severity may be set to be 0, 1, 2, 3, or 4 (maximum) when the number of auscultation positions at which abnormal lung sounds are heard is 0, 1 to 2, 3 to 4, 5 to 8, or 9 or more, respectively.

The severity information 254 shows a result determined by the severity determination unit 263 to be described below by using the analysis result information 252 and the severity determination information 253. FIG. 8 illustrates an example of the analysis result information 252. Referring to FIG. 8, in the severity information 254, severity identification information corresponding to the date/time when the severity determination unit 263 performed determination and the severity are associated with each other, for example.

In the field of severity in the severity information 154, a result determined by the severity determination unit 263 using the analysis result information 252 and the severity determination information 253 is recorded. That is, information indicating the above-described severity such as severity 0, severity 1, severity 2, and the like is recorded.

The personal condition information 255 is information representing the condition of the patient. The information included in the personal condition information 255 can be used for determining the severity by the severity determination unit 263. The personal condition information 255 is, for example, acquired in advance by a method such as being read from an external device or a storage medium via the data input/output function of the communication I/F unit 240 or the like, being input through operation of the operation input unit 220 by the user, or the like, and is stored in the storage unit 250. The personal condition information 255 may be appropriately updated by, for example, operating the operation input unit 220 each time analysis using the analysis device 200 is performed

FIG. 9 illustrates exemplary information included in the personal condition information 255. Referring to FIG. 9, in the personal condition information 255, condition identification information corresponding to the date/time that the condition information was input and the condition information are associated with each other, for example.

In the condition information field of the personal condition information 255, information representing the weight and the medication of the patient may be included. The information representing the medication may include information such as date/time that the patient took medicine last time, the type of medicine taken by the patient last time, and medication frequency. Further, the condition information field may include information representing blood pressure, pulse, subjective symptoms (short breath when goes out, edema, cough, anorexia, or the like), water intake, percutaneous arterial blood oxygen saturation (SPO2), or the like. The condition information field may also include informative matters from a doctor at the time of discharge from hospital or at the time of attending hospital.

The output information 256 represents the content output by the output unit 264 and a result of output by the output unit 264. FIG. 10 illustrates an example of the output information 256. Referring to FIG. 10, the output information 256 includes, for example, output identification information corresponding to the date/time of output by the output unit 265, severity, the output content, and the output result.

Here, the severity field indicates severity when the output unit 264 decides to perform output. Further, the output content field includes contents other than the severity output by the output unit 264. For example, the output content field may include at least one of lung sounds data, acquisition position, acquisition date/time, personal condition information, and the like. Further, the output result field includes information acquired from the processing device 300 as a result of output by the output unit 264. For example, the output result field includes information indicating a result of diagnosis performed according to the output such as follow-up observation, medication instruction, advice to seek diagnosis, and the like. The output result field may include information other than that illustrated above, such as a message from a home doctor.

The main information stored in the storage unit 250 is as described above. Note that various types of identification information such as data identification information, analysis result identification information, severity identification information, condition identification information, and output identification information may be different respectively, or may be common information such as information corresponding to date/time of the entire analysis, for example.

The arithmetic processing unit 260 has a microprocessor such as a CPU and the peripheral circuits thereof, and is configured to read and execute the program 257 from the storage unit 250 to allow the hardware and the program 257 to cooperate with each other to thereby implement the various processing units. The main processing units implemented by the arithmetic processing unit 260 include the lung sound acquisition unit 261, the abnormality detection unit 262, the severity determination unit 263, the output unit 264, and the receiving unit 265.

The lung sound acquisition unit 261 acquires digital time-series acoustic signals including lung sounds of a patient and other information. The lung sound acquisition unit 261 acquires digital time-series acoustic signals including lung sounds of a patient from the electronic stethoscope 210, in accordance with an instruction by a user input from the operation input unit 220 or the like. The lung sound acquisition unit 261 can also acquire information indicating date/time and the like, along with the digital time-series acoustic signals. Then, the lung sound acquisition unit 261 generates the lung sound data 251 as illustrated in FIG. 3 by using the acquired digital time-series acoustic signals and the other information, and stores it in the storage unit 250. As described above, the lung sound acquisition unit 261 may combine the data identification information and the information as illustrated in FIG. 2.

Note that any method may be used to acquire lung sounds of each auscultation position of a patient by an electronic stethoscope and record it in association with the auscultation position. For example, as described in Patent Literature 1, 4, 6 or the like, a method in which a guidance screen for guidance on the auscultation position to an operator who uses the electronic stethoscope 210 is shown on the screen display unit 230, or the like may be used. The lung sound acquisition unit 261 may instruct the breath timing to a patient by the method as described in Patent Literature 8.

The lung sound acquisition unit 261 may be configured to calculate an index value for the quality of lung sounds and give warning based on the calculated index value on the screen display unit 230 or the like. With such warning, a patient, a user, or the like can acquire lung sounds again after taking measures to reduce the background noise and/or increase the lung sounds.

An index value calculation process for the quality of lung sounds is performed by calculating and comparing the signal intensities after applying a predetermined filter, for example. For example, the lung sound acquisition unit 261 uses a bandpass filter to extract time-series acoustic signals in the frequency band of 100 Hz to about 2 kHz in which lung sounds of a patient are included, from the time-series acoustic signals output from the electronic stethoscope 210. Then, the lung sound acquisition unit 261 calculates the intensity of the lung sounds and the intensity of the background noise in the extracted time-series acoustic signals, and calculates the difference degree thereof as an index value of the quality of the lung sounds. For example, the lung sound acquisition unit 261 detects an inspiratory phase, an expiratory phase, and a pause phase from the time-series acoustic signals including lung sounds. Then, the lung sound acquisition unit 261 calculates the intensity of the time-series acoustic signals in the pause phase as the intensity of the background noise. As the intensity of the time-series acoustic signals, a root-mean-square of the amplitude value may be used for example. However, it is not limited thereto, and may be an amplitude or the like. Further, the lung sound acquisition unit 261 calculates a value obtained by subtracting the intensity of the background noise from the intensity of the time-series acoustic signals in the inspiratory phase and/or expiratory phase, as the intensity of the lung sounds. Then, the lung sound acquisition unit 261 uses the ratio of the calculated intensity of the lung sounds to the intensity of the background noise, as an index value of the quality of the lung sounds. Note that an index value of the quality of the lung sounds is not limited to that described above. It is also possible to use an S/N ratio calculated from the intensity of the lung sounds and the intensity of the background noise as an index value. Application of a filter may be omitted.

Note that the lung sound acquisition unit 261 can detect an expiratory phase and an inspiratory phase by comparing the time-series acoustic signals with a predetermined threshold. The lung sound acquisition unit 261 can also detect a predetermined period immediately before the detected inspiration start point of time as a pause phase. The lung sound acquisition unit 261 may detect the inspiratory phase, the expiratory phase, and the pause phase by using a method other than that illustrated above. For example, the lung sound acquisition unit 261 may be configured to acquire estimated probability of an inspiratory phase, an expiratory phase, and a pause phase for each section from a learning model, by inputting time-series acoustic signals including the lung sounds of the patient into a learning model having been learned through machine learning for estimating which section of the time-series acoustic signals including the lung sounds output from the electronic stethoscope 210 is an inspiratory phase, an expiratory phase, or a pause phase. A learning model can be generated in advance through machine learning using a machine learning algorism such as a neural network by using time-series acoustic signals including various lung sounds as teacher data.

Further, the lung sound acquisition unit 261 may remove the period of a pause phase and the background noise from the digital time-series acoustic signals including the lung sounds, and records, in the lung sound data 251, the digital time-series acoustic signals after removal of the period of the pause phase and the background noise, in association with the auscultation position. For example, the lung sound acquisition unit 261 divides the digital time-series acoustic signals including the lung sounds into two, that is, a section configured of an inspiratory phase and an expiratory phase immediately thereafter (hereinafter referred to as an inspiratory/expiratory section), and a section of a pause phase (hereinafter referred to as a pause section). Then, the lung sound acquisition unit 261 calculates the frequency spectrum of the inspiratory/expiratory section and the pause section by applying fast Fourier transform (FFT) to the digital time-series acoustic signals in each of the inspiratory/expiratory section and the pause section. Then, the lung sound acquisition unit 261 subtracts the frequency spectrum of the pause section from the frequency spectrum of the inspiratory/expiratory section. By the subtraction, the background noise included in the inspiratory phase and the expiratory phase is suppressed. Then, the lung sound acquisition unit 261 applies inverse frequency transform to the frequency spectrum of the inspiratory/expiratory section to thereby generate digital time-series acoustic signals after the removal of the noise in the inspiratory/expiratory section. Then, the lung sound acquisition unit 261 records the generated digital time-series acoustic signals after the removal of the noise in the inspiratory/expiratory section, in the lung sound data 251 in association with the auscultation position. Note that the lung sound acquisition unit 261 may remove the period of a pause phase from the digital time-series acoustic signals including the lung sounds of the auscultation position and not remove the background noise. In that case, the lung sound acquisition unit 261 divides the focused digital time-series acoustic signals including the lung sounds of the auscultation position into two, that is, the inspiratory/expiratory section and the pause section, and records the digital time-series acoustic signals in the inspiratory/expiratory section in the lung sound data 251, in association with the auscultation position.

The abnormality detection unit 262 detects abnormality from lung sound data of each auscultation position included in the lung sound data 251, and records the detection result in the analysis result information 252 in association with the auscultation position. For example, the abnormality detection unit 262 inputs the lung sound data into an abnormality detection model previously generated and stored, and acquires the probability that the lung sound data is abnormal lung sounds from the abnormality detection model. Then, the abnormality detection unit 262 compares the probability of abnormal lung sounds with a preset threshold. Then, when the probability exceeds the threshold, the abnormality detection unit 262 determines that the data is abnormal lung sounds. This means that the abnormality detection unit 262 detects abnormality. On the other hand, when the probability is equal to or smaller than the threshold, the abnormality detection unit 262 determines that the data is not abnormal lung sounds. Then, the abnormality detection unit 262 records the detection result in the analysis result information 252.

Note that the abnormality detection model can be generated in advance by, for example, generating teacher data by using a database in which abnormal sounds are collected, and learning characteristics of input sound data (input data) and determination criteria by using deep learning. For example, the abnormality detection unit 262 can use, for leaning and input data, a spectrum program in which sounds are applied with fast Fourier transform (FFT) or log-FFT for each certain section to be aligned in the time-series manner, and for deep learning, recurrent neural network (RNN) or convolutive neural network (CNN) can be used.

Further, the abnormality detection unit 262 may use a method in which a lung sound waveform is transformed into a short-time feature amount such as zero-cross coefficient or mel-frequency cepstral coefficient (MFCC) and abnormal sounds are detected by machine learning. For example, the abnormality detection unit 262 may perform modeling by mixed Gaussian distribution (GMM) at the time of learning, and check whether or not it matches the model at the time of detection. Further, the abnormality detection unit 262 may learn the identifying surface of an identifier such as a support vector machine (SVM) and uses the identifying surface to identify whether or not the input data corresponds to the abnormal sounds. The abnormality detection unit 262 may generate the feature amount by using the data itself like non-negative matrix factorization (NMF) or principal component analysis (PCA), other than the method of directly calculating the feature amount as described above.

Further, the abnormality detection unit 262 may detect abnormal sounds by the decision tree using statistical features of an input waveform such as long-time power distribution of input signals, distribution of component amount/component ratio of a specific frequency bin range, or the like. In that case, as items of the decision tree, the abnormality detection unit 262 may use statistical features (for example, when a process frame larger than 3σ is generated by Gaussian approximation), rather than a direct value (for example, when the power exceeds 20 mW for three consecutive frames). Further, the abnormality detection unit 262 may detect abnormal sounds by not using the input signal itself but modeling it through auto-regression (AR) process or the like and detecting abnormal sounds when some of the model parameters exceed a threshold. These methods may not include a learning process, but includes observation of abnormal sounds that are object signals in the configuration of the decision tree or determination of a threshold. Therefore, they are included in supervised learning for the sake of convenience.

Further, the abnormality detection unit 262 may be configured to learn an abnormality detection model by using past lung sound data of the patient and auscultation observations at the time of discharge from hospital or the like, for example. As lung sound data to be used for learning an abnormality detection model, in addition to the lung sound data of the patient at the time of discharge from hospital, it is also possible to use normal lung sound data of the patient before it or use normal lung sound data of a person other than the patient. By generating an abnormality detection model on the basis of lung sound data at the time of discharge from hospital, it is possible to perform detection in consideration of the condition of the patient at the time of discharge from hospital.

Note that an abnormality detection model may be generated for each auscultation position, or may be shared by a plurality of auscultation positions. Further, abnormality detection models may be a plurality of models that are machine-learned from different viewpoints. For example, abnormality detection models may include a model in which lung sounds of the same auscultation position is divided into a lung sound portion of the inspiratory phase, a lung sound portion of the expiratory phase, and a portion other than those (that is, pause phase) on the basis of the breath timing, and learning is performed by using the lung sound portion of the inspiratory phase, and a model obtained through learning by using the lung sound portion of the expiratory phase.

The severity determination unit 263 determines the severity, on the basis of the analysis result of each auscultation position indicated by the analysis result information 252, and the severity determination information 253. Then, the severity determination unit 263 stores the determined severity in association with the severity identification information in the storage unit 250 as the severity information 254.

For example, the severity determination unit 263 refers to the analysis result information 252 to specify the auscultation position in which abnormality is detected. Then, the severity determination unit 263 refers to the severity determination information 253 to determine the severity corresponding to the specified result.

As described above, the severity determination information 253 can be created according to the condition of the patient at the time of discharge from hospital. In the case where the severity determination information 253 is created as described above, the severity determination unit 263 may also refer to the severity determination information 253 corresponding to the condition of the patient at the time of discharge from hospital to determine the severity corresponding to the condition of the patient at the time of discharge from hospital.

Moreover, the severity determination unit 263 may be configured to revise the determined severity, by referring to the personal condition information 255 when determining the severity. For example, there is an observation that weight increases due to accumulation of water in lungs when the heart failure gets worse. Therefore, the severity determination unit 263 can revise the determined severity on the basis of the weight of the patient included in the personal condition information 256. For example, when a predetermined condition is satisfied such as the weight increasing by 3 kg in a week, the severity determination unit 263 can revise the severity by a set value such as incrementing the determined severity by 1. Note that the severity determination unit 263 may perform revision other than that described above such as incrementing the severity by 2 when the weight increases by 5 kg in a week. Further, the severity determination unit 263 may perform revision using the personal condition information 255 other than that described above, by, for example, incrementing the severity when the blood pressure is lowered by a predetermined value or more or the pulse exceeds a predetermined number. The severity determination unit 263 may directly determine the severity in consideration of the personal condition information 255 by referring to the severity determination information 253 in which severity is set according to the information that may be included in the personal condition information 256.

The output unit 264 decides whether or not to output predetermined information to the processing device 300 on the basis of the result of determination by the severity determination unit 263. Then, the output unit 264 outputs the predetermined information according to the result of determination. The output unit 264 also stores the output severity and the other output contents in association with the output identification information, in the storage unit. The output by the output unit 264 to the processing device 300 may be realized by using at least one communication method such as email, messaging function of groupware, and business chat.

For example, the output unit 264 checks whether or not the severity determined by the severity determination unit 263 exceeds an output threshold. When the severity exceeds the output threshold, the output unit 264 decides to output to the processing device 300. Then, the output unit 264 outputs the predetermined information to the processing device 300.

Here, the information output by the output unit 264 is previously determined for example. For example, the information output by the output unit 264 includes at least one of severity determined by the severity determination unit 263, lung sound data and auscultation position included in the lung sound data 251, information indicating the date/time on which the lung sound data was acquired, information such as weight and medication included in the personal condition information 255, and the like. The output unit 264 may output information other than that mentioned above as examples, such as information included in the analysis result information 252, frequency spectrum calculated by applying first Fourier transform (FFT) to the lung sound data, and the like.

Note that the information output by the output unit 264 may be only lung sound data in which abnormality is detected by the abnormality detection unit 262 of the lung sound data included in the lung sound data 251, or the entire lung sound data included in the lung sound data 251. Besides the lung sound data acquired at the current analysis, the output unit 264 may output past lung sound data such as lung sound data in the last two weeks, lung sound data in which no abnormality was detected by the abnormality detection unit 262, or the like. The output unit 264 may output frequency spectrum calculated from the lung sound data acquired at the current analysis and frequency spectrum calculated from the lung sound data in which no abnormality was detected by the abnormality detection unit 262. The output unit 264 may output average frequency spectrum of the patient whose diagnosis result is an advice to seek diagnosis, besides the frequency spectrum as described above. Further, the information such as weight and medication included in the personal condition information of the information output by the output unit 264 may also include past information such as a change of weight in the last two weeks or medication in the last two weeks.

Further, the output unit 264 may change the information to be output, corresponding to the severity determined by the severity determination unit 263. For example, the output unit 264 may set a difference in information to be output corresponding to the severity, such as increasing the information to be output as the severity is higher, outputting only lung sound data when the severity is equal to or smaller than a transmission threshold while outputting lung sound data and information included in the personal condition information 255 when the severity exceeds the transmission threshold.

Further, the processing device 300 that is an output destination of the output unit 264 is determined previously, for example. For example, the output unit 264 has information in which an identifier of a user of the analysis device 200 or a patient and the processing device 300 held by a home doctor of the patient or the like are associated with each other. Then, the output unit 264 refers to the information to thereby output predetermined information to the processing device 300 held by a home doctor of the patient or the like.

Note that the output unit 264 may refer to the past output result included in the output information 256 when deciding whether or not to output predetermined information to the processing device 300. For example, in the case where the output information 256 includes information in which the severity is the same as the severity determined by the severity determination unit 263 (or the difference is equal to or smaller than a predetermined value) and the output result is follow-up observation, the output unit 264 can decide not to output to the processing device 300 although the severity exceeds the output threshold. When the output information 256 includes the information in which the severity is the same as the severity determined by the severity determination unit 263 and the output result is follow-up observation, the output unit 264 may decide whether or not to output, on the basis of a result of spectrum analysis. For example, the output unit 264 calculates the frequency spectrum by applying fast Fourier transform (FFT) to the lung sound data of the time that the output result is follow-up observation and the current lung sound data. Then, the output unit 264 compares the calculated frequency spectrum to thereby decide whether or not to transmit predetermined information to the processing device 300. For example, the output unit 264 determines to transmit information to the processing device 300 when the difference in the frequency spectrum is equal to or smaller than a predetermined value. Further, when the output information 256 includes the information in which the severity is the same as the severity determined by the severity determination unit 263 and the output result is follow-up observation, the output unit 264 may decide whether or not to output according to the content of the lung sound data such as the number and frequency of rales. For example, when the difference in the lung sound data such as the number and frequency of rales is equal to or smaller than a predetermined value, the output unit 264 may decide not to output the information.

The receiving unit 265 receives a result of output by the output unit 264 from the processing device 300. Then, the receiving unit 265 updates the output result field of the output information 256 on the basis of the received information.

For example, the receiving unit 265 receives, from the processing device 300, information representing a result of diagnosis by a home doctor having the processing device 300 corresponding to the content of output by the output unit 264 such as follow-up observation, a medication instruction, an advice to seek diagnosis, or the like. Then, the receiving unit 265 updates the output information 256 by storing the received information in the output result field of the output information 256.

The processing device 300 is an information processing device that receives information output by the output unit 264 of the analysis device 200. The processing device 300 is installed in, for example, a hospital where a home doctor works. The processing device 300 may be a smartphone, a tablet terminal, a personal digital assistant (PDA), a laptop personal computer, or the like, for example. The processing device 300 may be one other than that illustrated above as an example.

FIG. 11 illustrates an exemplary configuration of the processing device 300. Referring to FIG. 11, the processing device 300 includes, for example, an operation input unit 310, a screen display unit 320, a communication I/F unit 330, a storage unit 340, and an arithmetic processing unit 350, as main constituent elements. Note that the configurations of the operation input unit 310, the screen display unit 320, and the communication I/F unit 330 may be the same as the configurations included in the analysis device 200. Therefore, the description thereof is omitted.

The storage unit 340 is a storage device such as a hard disk or a memory. The storage unit 340 stores therein processing information and a program 343 required for various types of processing performed in the arithmetic processing unit 350. The program 343 is read and executed by the arithmetic processing unit 350 to thereby implement various processing units. The program 343 is read in advance from an external device or a storage medium via the data input/output function of the communication I/F unit 330 and the like, and is stored in the storage unit 340. The main information to be stored in the storage unit 340 includes acquired information 341 and diagnosis result 342, for example.

The acquired information 341 includes information output by the analysis device 200. That is, the acquired information 341 includes information according to the output by the output unit 264. The information included in the acquired information 341 can be output to the screen display unit 320.

The diagnosis result 342 represents a result of diagnosis by a home doctor or the like corresponding to the acquired information 341. For example, the diagnosis result 342 represents information showing a result of diagnosis by a home doctor having the processing device 300 corresponding to the acquired information 341, such as follow-up observation, a medication instruction, an advice to seek diagnosis, or the like.

The arithmetic processing unit 350 has a microprocessor such as a CPU and the peripheral circuits thereof, and is configured to read and execute the program 343 from the storage unit 340 to allow the hardware and the program 343 to cooperate with each other to thereby implement the various processing units. The main processing units implemented by the arithmetic processing unit 350 include an acquisition unit 351, a diagnosis result input unit 352, and an output unit 353.

The acquisition unit 351 acquires information output by the output unit 264 of the analysis device 200. Then, the acquisition unit 351 stores the acquired information in the storage unit 340 as the acquired information 341.

The diagnosis result input unit 352 acquires a diagnosis result of a home doctor or the like with respect to the acquired information 341 stored by the acquisition unit 351. In response to an operation performed on the operation input unit 310 or the like, the diagnosis result input unit 352 acquires information indicating a result of diagnosis by a home doctor corresponding to the acquired information 341 such as follow-up observation, a medication instruction, an advice to seek diagnosis, or the like. Then, the diagnosis result input unit 352 stores the acquired information in the storage unit 340 as the diagnosis result 342.

The output unit 353 outputs the diagnosis result 342 to the analysis device 200. For example, when the diagnosis result input unit 352 stores a new diagnosis result 342, the output unit 353 transmits information showing the new diagnosis result to the analysis device 200.

For example, as described above, the processing device 300 acquires information showing the diagnosis result corresponding to the output by the output unit 264, and sends back the information showing the diagnosis result to the analysis device 200. Note that the information showing the diagnosis result may include information other than that described above as an example, such as a message from a home doctor and the like.

Next, an exemplary operation of the analysis device 200 will be described with reference to FIG. 12.

Referring to FIG. 12, the lung sound acquisition unit 261 acquires digital time-series acoustic signals including lung sounds of a patient and the like, for each auscultation position (step S101). Then, the lung sound acquisition unit 261 generates the lung sound data 251 as illustrated in FIG. 3 by using the acquired digital time-series acoustic signals and the other information, and stores it in the storage unit 250. As described above, the lung sound acquisition unit 261 may combine data identification information and the information as illustrated in FIG. 3.

When acquiring digital time-series acoustic signals and the like, the lung sound acquisition unit 261 may calculate an index value for the quality of lung sounds and give alarm based on the calculated index value on the screen display unit 230. The lung sound acquisition unit 261 may generate lung sound data 251 by using data after performing processing such as removal of time-series acoustic signals in a period of a pause phase, noise removal, and application of respiration timing, and store it in the storage unit 250.

The abnormality detection unit 262 detects abnormality from lung sound data of each auscultation position included in the lung sound data 251, and records the detection result in the analysis result information 252 in association with the auscultation position (step S102). For example, the abnormality detection unit 262 inputs the lung sound data into the abnormality detection model previously generated and stored, and acquires the probability that the lung sound data is abnormal lung sounds from the abnormality detection model. Then, the abnormality detection unit 262 compares the probability of abnormal lung sounds with a preset threshold. Then, when the probability exceeds the threshold, the abnormality detection unit 262 determines that the data is abnormal lung sounds. This means that the abnormality detection unit 262 detects abnormality. On the other hand, when the probability is equal to or smaller than the threshold, the abnormality detection unit 262 determines that the data is not abnormal lung sounds. Then, the abnormality detection unit 262 records the detection result in the analysis result information 252.

The severity determination unit 263 determines the severity on the basis of the analysis result of each auscultation position indicated by the analysis result information 252, and the severity determination information 253 (step S103). Then, the severity determination unit 263 stores the determined severity in the storage unit 250 as the severity information 254 in association with the severity identification information.

Note that the severity determination unit 263 may determine the severity corresponding to the condition of the patient at the time of discharge from hospital, by referring to the severity determination information 253 corresponding to the condition of the patient at the time of discharge from hospital. Moreover, the severity determination unit 263 may revise the determined severity by referring to the personal condition information 255 when determining the severity. The severity determination unit 263 may directly determine the severity in consideration of the personal condition information 255 by referring to the severity determination information 253 in which severity is set corresponding to the information that may be included in the personal condition information 255.

The output unit 264 determines whether or not to output predetermined information to the processing device 300 on the basis of the result of determination by the severity determination unit 263. For example, the output unit 264 checks whether or not the severity determined by the severity determination unit 263 exceeds an output threshold (step S104).

When the severity exceeds the output threshold (step S104, Yes), the output unit 264 checks whether or not the output information 256 satisfies the conditions (step S105). When the output information 256 satisfies the condition (step S105, Yes), the output unit 264 decides to output the predetermined information to the processing device 300, and transmits the predetermined information to the processing device 300 (step S106). Further, the output unit 264 stores the output information in the storage unit 250 as the output information 256. Note that the conditions may include whether the output information includes information in which the severity is the same as the severity determined by the severity determination unit 263 and the output result field is follow-up observation, and in the case where such information is included, a difference in frequency spectrum, or the like.

The receiving unit 265 receives a result of output by the output unit 264 from the processing device 300 (step S107). Then, the receiving unit 265 stores the output result field of the output information 256 on the basis of the received information (step S108).

When the severity is equal to or smaller than the output threshold (step S104, No), or when the output information 256 does not satisfy the condition (step S105, No), the output unit 264 decides not to perform output to the processing device 300.

The exemplary operation of the analysis device 200 is as described above. Note that the processing of step S105 may be omitted.

As described above, the analysis device 200 includes the abnormality detection unit 262, the severity determination unit 263, and the output unit 264. According to such a configuration, the output unit 264 can decide whether or not to perform output to the processing device 300 on the basis of the severity determined by the severity determination unit 263 on the basis of the detection result by the abnormality detection unit 262. As a result, when the necessity of sharing information is high such as the case where the severity is high, it is possible to share the examination result with a medical professional. That is, according to the above-described configuration, it is possible to realize efficient information sharing.

It is also assumed that acquisition of lung sound data using the electronic stethoscope 210 may be terminated due to the circumstances of the patient or the like. In that case, the abnormality detection unit 262 performs analysis only on the auscultation positions in which lung sound data has been acquired. The severity determination unit 263 may be configured to decide whether or nor to perform determination of severity, on the basis of the number of auscultation positions on which the abnormality detection unit 262 has performed analysis. For example, when the number of auscultation positions in which lung sound data has not been acquired and analysis of whether or not the data is abnormal lung sounds has not been performed is smaller than a preset threshold, the severity determination unit 263 does not calculate severity and can display that the analysis is terminated in error, on the screen display unit 230. This is because not to give erroneous information to the operator or the like. On the contrary, when the number of auscultation positions is equal to or larger than a preset threshold, the severity determination unit 263 assumes that no abnormal lung sound was detected at auscultation positions in which analysis of whether or not the lung sounds are abnormal has not been performed, and can calculate the severity. In that case, the severity determination unit 263 may hold the calculated severity as the most optimistic value. That is, when the calculated severity is the severity 1, it is not held as “severity 1” but can be held as “severity 1 or higher” or “at least severity 1”. Note that any threshold may be set.

Further, the analysis device 200 may be configured to determine on which auscultation position to be focused, on the basis of the auscultation position at which abnormality was detected in the past. For example, the lung sound acquisition unit 261 of the analysis device 200 can calculate the abnormality detection frequency for each auscultation position on the basis of the past analysis result information 252. Further, the lung sound acquisition unit 261 can give guidance to acquire lung sound data in the descending order of the calculated abnormality detection frequency. In the case of acquiring lung sound data in the descending order of the abnormality detection frequency, the threshold to be used for determining whether or not the severity determination unit 263 calculates the severity may be smaller, compared with the case of not acquiring lung sound data in the descending order of the abnormal detection frequency.

Further, the analysis device 200 may be configured to, when a patient or the like confirms the information indicating the diagnosis result received by the receiving unit 265, transmit information indicating that confirmation has been made, to the processing device 300. With such a configuration, a home doctor or the like who operates the processing device 300 can easily check whether or not a patient or the like confirms the information indicating the result.

### Second Exemplary Embodiment

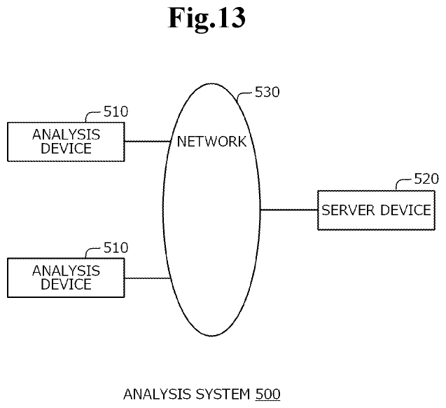

FIG. 13 is a block diagram of an analysis system 500 according to a second exemplary embodiment of the present invention. Referring to FIG. 13, the analysis system 500 is configured of a plurality of analysis devices 510 and a server device 520. The analysis devices 510 and the server device 520 are communicably connected with each other over a network 530 such as the Internet.

The analysis device 510 is an information processing device that outputs an instruction corresponding to a result of analyzing lung sounds. The analysis device 510 may be a smartphone, a tablet terminal, a PDA, a laptop personal computer, or the like, but is not limited thereto. The analysis device 510 includes an electronic stethoscope, a communication I/F unit, an operation input unit, a screen display unit, a storage unit, and an arithmetic processing unit that are not illustrated.

The server device 520 is a computer that provides, to the analysis devices 510, various services required for lung sound analysis over the network 530. For example, the server device 520 stores therein at least part of the lung sound data 251, the analysis result information 252, the severity determination information 253, the severity information 254, the personal condition information 255, the output information 256, and the program 257 illustrated in FIG. 2, and provides them to the analysis device 510 over the network 530. Therefore, the analysis device 510 is not needed to store at least part of the lung sound data 251, the analysis result information 252, the severity determination information 253, and the severity information 254, the personal condition information 255, the output information 256, and the program 157 in the storage unit as compared with the analysis device 200 of FIG. 2, so that the memory capacity can be reduced.

Further, the server device 520 can also provide at least part of the functions of the lung sound acquisition unit 261, the abnormality detection unit 262, the severity determination unit 263, the output unit 264, and the receiving unit 265 illustrated in FIG. 2, to the analysis device 510 over the network 530. That is, the server device 520 executes at least a part of the processing illustrated in FIG. 12 in place of the analysis device 510. Therefore, in the analysis device 510, the configuration of the arithmetic processing unit 260 can be simplified as compared with the analysis device 200 of FIG. 2.

For example, the function as the analysis device 200 may be implemented by the analysis system 500 or the like, as described above.

### Third Exemplary Embodiment

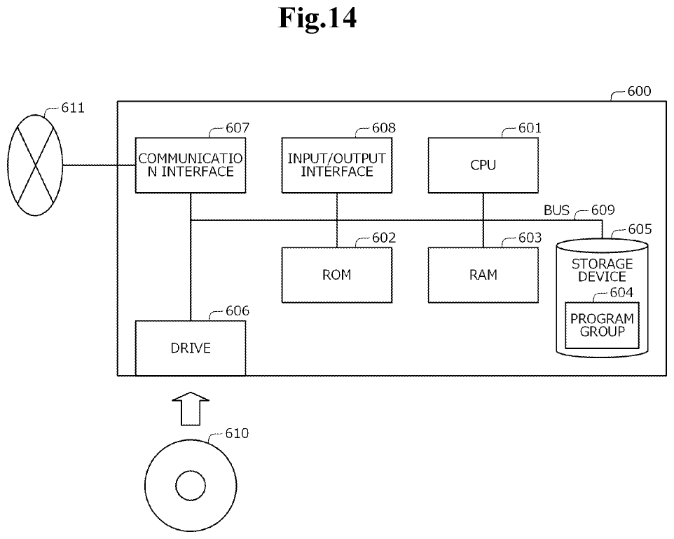

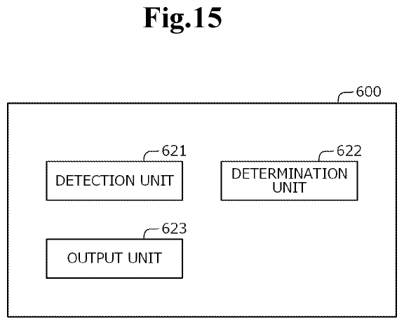

Next, a third exemplary embodiment of the present invention will be described with reference to FIGS. 14 and 15. In the third exemplary embodiment, a configuration of an analysis device 600 will be described.

FIG. 14 illustrates an exemplary hardware configuration of the analysis device 600. Referring to FIG. 14, the analysis device 600 includes a hardware configuration as described below, as an example.
Central Processing Unit (CPU) 601 (arithmetic device)
Read Only Memory (ROM) 602 (storage device)
Random Access Memory (RAM) 603 (storage device)
Program group 604 to be loaded to the RAM 603
Storage device 605 storing therein the program group 604
Drive 606 that performs reading and writing on a storage medium 610 outside the information processing device
Communication interface 607 connecting to a communication network 611 outside the information processing device
Input/output interface 608 for performing input/output of data
Bus 609 connecting the respective constituent elements

Further, the analysis device 600 can realize the functions as a detection unit 621, a determination unit 622, and an output unit 623 illustrated in FIG. 15 through acquisition and execution of the program group 604 by the CPU 601. Note that the program group 604 is stored in the storage device 605 or the ROM 602 in advance, and is loaded to the RAM 603 by the CPU 601 as needed. Further, the program group 604 may be provided to the CPU 601 via the communication network 611, or may be stored on a storage medium 610 in advance and read out by the drive 606 and supplied to the CPU 601.

FIG. 14 illustrates an exemplary hardware configuration of the analysis device 600. The hardware configuration of the analysis device 600 is not limited to that described above. For example, the analysis device 600 may be configured of part of the configuration described above, such as without the drive 606.

The detection unit 621 detects abnormality in lung sounds for each auscultation position on the basis of time-series acoustic signals including lung sounds of each auscultation position.

The determination unit 622 determines severity of the heart failure of a patient on the basis of a detection result of abnormality in the lung sounds of each auscultation position detected by the detection unit 621 and the condition information representing the condition of the patient.

The output unit 623 decides whether or not to output predetermined information to an external device on the basis of the result of determination by the determination unit 622, and performs output corresponding to the result of decision.

As described above, the analysis device 600 includes the detection unit 621, the determination unit 622, and the output unit 623. According to such a configuration, the output unit 623 can decide whether or not to perform output to the processing device 300 on the basis of the severity determined by the determination unit 622 based on a detection result by the detection unit 621. As a result, it is possible to share the examination result with a medical professional in the case where it is necessary to share information such as the severity being high. That is, according to the above-described configuration, efficient information sharing can be realized.

Note that the analysis device 600 as described above can be realized by incorporation of a predetermined program into an information processing device such as the analysis device 600. Specifically, a program that is another aspect of the present invention is a program for implementing, in an information processing device, a detection unit that detects abnormality in lung sounds for each auscultation position on the basis of time-series acoustic signals including lung sounds of each auscultation position, a determination unit that determines severity of heart failure of a patient on the basis of a detection result of abnormality in the lung sounds of each auscultation position detected by the detection unit and condition information representing the condition of the patient, and an output unit that decides whether or not to output predetermined information to an external device on the basis of a result of determination by the determination unit, and performs output corresponding to a result of decision.

Further, an analysis method to be implemented by an information processing device such as the analysis device 600 is a method including, by the information processing device, detecting abnormality in lung sounds for each auscultation position on the basis of time-series acoustic signals including lung sounds of each auscultation position, determining severity of heart failure of a patient on the basis of a detection result of abnormality in the lung sounds of each detected auscultation position and condition information representing the condition of the patient, and deciding whether or not to output predetermined information to an external device on the basis of a result of determination and performing output corresponding to a result of decision.

Since the invention of a program (or storage medium) or an analysis method having the above-described configuration also exhibits the same actions and effects as those of the analysis device 600, the above-described object of the present invention can be achieved.

While the present invention has been described with reference to the exemplary embodiments described above, the present invention is not limited to the above-described embodiments. The form and details of the present invention can be changed within the scope of the present invention in various manners that can be understood by those skilled in the art, such as a combination of various modifications.

### INDUSTRIAL APPLICABILITY

The present invention is applicable to a device and a system for analyzing lung sounds of a person, and in particular, applicable to a device and a system for detecting, in an early stage, exacerbation of heart failure of a patient who received heart failure treatment and was discharged from hospital to prevent re-hospitalization.

The whole or part of the exemplary embodiments disclosed above can be described as, but not limited to, the following supplementary notes.

### (Supplementary Note 1)

An analysis device comprising:
a detection unit that detects abnormality in lung sounds for each of auscultation positions on a basis of time-series acoustic signals including lung sounds of each of the auscultation positions;
a determination unit that determines severity of heart failure of a patient on a basis of a detection result of abnormality in the lung sounds of each of the auscultation positions detected by the detection unit and condition information representing a condition of the patient; and
an output unit that decides whether or not to output predetermined information to an external device on a basis of a result of the determination by the determination unit, and performs output corresponding to a result of the decision.

### (Supplementary Note 2)

The analysis device according to supplementary note 1, wherein
the output unit decides whether or not to output the predetermined information to the external device on a basis of whether or not the severity determined by the determination unit exceeds an output threshold.

### (Supplementary Note 3)

The analysis device according to supplementary note 1 or 2, wherein
the output unit decides whether or not to output the predetermined information to the external device on a basis of information received from the external device according to a result output by the output unit in past.

### (Supplementary Note 4)

The analysis device according to supplementary note 3, wherein
the output unit decides whether or not to output the predetermined information to the external device on a basis of information received from the external device according to a result output in past having severity that is same as the severity determined by the determination unit.

### (Supplementary Note 5)

The analysis device according to supplementary note 3 or 4, wherein
the output unit decides whether or not to output the predetermined information to the external device by using frequency spectrum calculated from the time-series acoustic signals, in a case where information including follow-up observation has been received as a result of output from the external device for severity corresponding to the severity determined by the determination unit.

### (Supplementary Note 6)

The analysis device according to supplementary note 5, wherein
the output unit decides whether or not to output the predetermined information to the external device on a basis of a comparison result between frequency spectrum calculated from the time-series acoustic signals used for the detection by the detection unit and frequency spectrum calculated from past time-series acoustic signals.

### (Supplementary Note 7)

The analysis device according to any one of supplementary notes 1 to 6, wherein
the predetermined information includes the severity determined by the determination unit and the time-series acoustic signals used for the detection by the detection unit.

### (Supplementary Note 8)

The analysis device according to supplementary note 7, wherein
the predetermined information includes at least one of information representing weight of the patient and information representing medication.

### (Supplementary Note 9)

The analysis device according to supplementary note 7 or 8, wherein
the predetermined information includes frequency spectrum calculated from the time-series acoustic signals used for the detection by the detection unit.

### (Supplementary Note 10)

The analysis device according to any one of supplementary notes 1 to 9, further comprising
a receiving unit that receives information indicating a result of diagnosis according to a result output by the output unit.

### (Supplementary Note 11)

The analysis device according to any one of supplementary notes 1 to 10, wherein
the external device is a device used by a home doctor.

### (Supplementary Note 12)

An analysis method comprising, by an information processing device:
detecting abnormality in lung sounds for each of auscultation positions on a basis of time-series acoustic signals including lung sounds of each of the auscultation positions;
determining severity of heart failure of a patient on a basis of a detection result of abnormality in the lung sounds of each of the auscultation positions detected and condition information representing a condition of the patient; and
deciding whether or not to output predetermined information to an external device on a basis of a result of the determination, and performing output corresponding to a result of the decision.

### (Supplementary Note 13)

A computer-readable medium storing thereon a program for causing an information processing device to implement:
a detection unit that detects abnormality in lung sounds for each of auscultation positions on a basis of time-series acoustic signals including lung sounds of each of the auscultation positions;
a determination unit that determines severity of heart failure of a patient on a basis of a detection result of abnormality in the lung sounds of each of the auscultation positions detected by the detection unit and condition information representing a condition of the patient; and
an output unit that decides whether or not to output predetermined information to an external device on a basis of a result of the determination by the determination unit, and performs output corresponding to a result of the decision.

### REFERENCE SIGNS LIST

100 analysis system

200 analysis device

210 electronic stethoscope

220 operation input unit

230 screen display unit

240 communication I/F unit

250 storage unit

151 lung sound data

152 analysis result information

153 severity determination information

154 severity information

255 personal condition information

256 output information

257 program

260 arithmetic processing unit

261 lung sound acquisition unit

262 abnormality detection unit

263 severity determination unit

264 output unit

265 receiving unit

300 processing device

310 operation input unit

320 screen display unit

330 communication I/F unit

340 storage unit

341 acquired information

342 diagnosis result

350 arithmetic processing unit

351 acquisition unit

352 diagnosis result input unit

353 output unit

400 network

500 analysis system

510 analysis device

520 server device

530 network

600 analysis device

601 CPU

602 ROM

603 RAM

604 program group

605 storage device

606 drive

607 communication interface

608 input/output interface

609 bus

610 storage medium

611 communication network

621 detection unit

622 determination unit

623 output unit

What is claimed is:

1. An analysis device comprising:
at least one memory configured to store instructions; and
at least one processor configured to execute instructions to:
detect abnormality in lung sounds for each of auscultation positions on a basis of time-series acoustic signals including lung sounds of each of the auscultation positions;
determine severity of heart failure of a patient on a basis of a detection result of abnormality in the lung sounds of each of the auscultation positions and condition information representing a condition of the patient; and
decide whether or not to output predetermined information to an external device on a basis of a result of the determination and perform output corresponding to a result of the decision.

2. The analysis device according to claim 1, wherein the at least one processor is configured to execute the instructions to
decide whether or not to output the predetermined information to the external device on a basis of whether or not the severity exceeds an output threshold.

3. The analysis device according to claim 1, wherein the at least one processor is configured to execute the instructions to
decide whether or not to output the predetermined information to the external device on a basis of information received from the external device according to a result output in past.

4. The analysis device according to claim 3, wherein the at least one processor is configured to execute the instructions to
decide whether or not to output the predetermined information to the external device on a basis of information received from the external device according to a result output in past having severity that is same as the determined severity.

5. The analysis device according to claim 3, wherein the at least one processor is configured to execute the instructions to
decide whether or not to output the predetermined information to the external device by using frequency spectrum calculated from the time-series acoustic signals, in a case where information including follow-up observation has been received as a result of output from the external device for severity corresponding to the determined severity.

6. The analysis device according to claim 5, wherein the at least one processor is configured to execute the instructions to
decide whether or not to output the predetermined information to the external device on a basis of a comparison result between frequency spectrum calculated from the time-series acoustic signals used for the detection and frequency spectrum calculated from past time-series acoustic signals.

7. The analysis device according to claim 1, wherein
the predetermined information includes the determined severity and the time-series acoustic signals used for the detection.

8. The analysis device according to claim 7, wherein
the predetermined information includes at least one of information representing weight of the patient and information representing medication.

9. The analysis device according to claim 7, wherein
the predetermined information includes frequency spectrum calculated from the time-series acoustic signals used for the detection.

10. The analysis device according to claim 1, wherein the at least one processor is configured to execute the instructions to
receive information indicating a result of diagnosis according to an output result.

11. The analysis device according to claim 1, wherein
the external device is a device used by a home doctor.

12. An analysis method comprising, by an information processing device:
detecting abnormality in lung sounds for each of auscultation positions on a basis of time-series acoustic signals including lung sounds of each of the auscultation positions;
determining severity of heart failure of a patient on a basis of a detection result of abnormality in the lung sounds of each of the auscultation positions detected and condition information representing a condition of the patient; and
deciding whether or not to output predetermined information to an external device on a basis of a result of the determination, and performing output corresponding to a result of the decision.

13. A non-transitory computer-readable medium storing thereon a program comprising instructions for causing an information processing device to implement:
a detection unit that detects abnormality in lung sounds for each of auscultation positions on a basis of time-series acoustic signals including lung sounds of each of the auscultation positions;
a determination unit that determines severity of heart failure of a patient on a basis of a detection result of abnormality in the lung sounds of each of the auscultation positions detected by the detection unit and condition information representing a condition of the patient; and
an output unit that decides whether or not to output predetermined information to an external device on a basis of a result of the determination by the determination unit, and performs output corresponding to a result of the decision.
